(19) United States
(12) Patent Application Publication
(10) Pub. No.: US 2024/0110933 A1
(43) Pub. Date: Apr. 4, 2024

(54) METHOD FOR DETECTING AMMONIA NITROGEN CONTENT BY USING NITRIFICATION BIOLOGICAL REACTION

(71) Applicant: WUYI UNIVERSITY, Jiangmen (CN)

(72) Inventors: Changyu LIU, Jiangmen (CN); Liang WANG, Jiangmen (CN); Jianbo JIA, Jiangmen (CN); Xiaolong XU, Jiangmen (CN); Mengchen ZHANG, Jiangmen (CN)

(21) Appl. No.: 18/329,652

(22) Filed: Jun. 6, 2023

(30) Foreign Application Priority Data
Sep. 30, 2022 (CN) 2022112135860
Apr. 28, 2023 (CN) 202310487367X

(51) Int. Cl. G01N 33/84

(52) U.S. Cl. CPC G01N 33/84; G01N 2520/00

(57) ABSTRACT

Provided is a method for detecting ammonia nitrogen content by using a nitrification biological reaction. In the present disclosure, the nitrifying microorganisms have high specificity for ammonia nitrogen, a metabolism and oxygen consumption capacity closely related to the ammonia nitrogen concentration, high accuracy and high sensitivity of ammonia nitrogen detection, and are not easily disturbed by water body chromaticity, suspended matters, and the like. In addition, the activity of the nitrification microorganism membrane reactor can be kept stable for a long term, and the stability of a sensor is good for long-term use. In the present disclosure, with a mono-component or multi-component solution containing inorganic nitrogen and inorganic carbon as a nitrification nutrient solution, a nitrification microorganism membrane with strong adaptability to the environment, a stable structure, a high selectivity for ammonia nitrogen, and an ability of efficiently degrading ammonia nitrogen can be obtained.

### CROSS-REFERENCE TO RELATED APPLICATION

This application claims priority of the Chinese patent application No. 2022112135860 filed Sep. 30, 2022, and Chinese patent application No. 202310487367X filed Apr. 28, 2023. The entire contents of these application are incorporated herein by reference.

### TECHNICAL FIELD

The present disclosure relates to the technical field of water body detection, in particular to a method for detecting ammonia nitrogen content by using a nitrification biological reaction.

### BACKGROUND

Ammonia nitrogen is one of the main pollutants resulting from eutrophication of a water body. Excessive ammonia nitrogen content in the water body may be harmful to the growth of fish and aquatic organisms, and ammonia nitrogen may also cause varying extents of damage to the human body after entering the human body through routes such as food chains. Therefore, the detection of ammonia nitrogen in water is of great significance for water resources protection and ecosystem improvement.

Existing detection methods for ammonia nitrogen mainly include: 1) Nessler's reagent spectrophotometry (also known as Nessler's reagent colorimetry), 2) salicylic acid-hypochlorite spectrophotometry and 3) ammonia gas-sensitive electrode method.

For Nessler's reagent spectrophotometry, under alkaline conditions, ammonia reacts with Nessler's reagent to generate a reddish-brown compound. In a certain concentration range, since the concentration of the generated reddish-brown compound is linear with the absorbance, the ammonia nitrogen content in a water sample can be quantitatively calculated according to the measured absorbance. However, the absorbance is easily disturbed by factors such as temperature, chromaticity, turbidity, and color development time, making it unsuitable for heavily polluted water sources and require relatively high professional skills to operate. In addition, the mercury iodide reagent used in this method is toxic and may cause secondary pollution to the environment if the waste liquid is not treated properly after measurement, thereby posing certain limitations on on-site measurements.

For salicylic acid-hypochlorite spectrophotometry, under the action of sodium nitroprusside, ammonium ions in water, together with salicylic acid and hypochlorite, form a blue-green stable compound, which has a strong absorbance at about 700 nm that is linear with the ammonia nitrogen concentration. However, the absorbance is easily disturbed by metal ions, turbidity, and chromaticity in the water body, and this method requires higher professional operation skills of researchers.

For ammonia gas-sensitive electrode method, the principle is that under the condition of pH>11, ammonium ions are transformed into ammonia, and ammonia is transferred through a hydrophobic membrane of an ammonia-sensitive electrode, resulting in the change of the electromotive potential of the ammonia-sensitive electrode. Since this potential difference is linear with the logarithm of the concentration of ammonia nitrogen in the water sample, the concentration of ammonia nitrogen can be measured according to the change of the electromotive potential. However, sodium hydroxide reagent is strongly corrosive, which brings a lot of inconvenience to the equipment anti-corrosion work. Moreover, the waste liquid after detection cannot be directly discharged into natural water bodies, and the electrode has a short service life (3-6 months), poor stability and reliability, and a low measurement accuracy.

Since the existing testing methods have many disadvantages, such as high interference factors, poor measurement stability and accuracy, and easily cause environmental pollution, there is a need to provide a rapid, accurate and environmentally friendly high-detection-stability method for measuring ammonia nitrogen content in a water body.

### SUMMARY

An object of the present disclosure is to provide a novel method for detecting ammonia nitrogen content by using a nitrification biological reaction, in order to overcome the defects of existing ammonia nitrogen detection methods having relatively poor measurement stability and accuracy and easily causing environmental pollution. In the detection method of the present disclosure, the ammonia nitrogen content in a water body is indirectly determined by using the indirect relationship between the amount of ammonia consumed during nitrification reactions in microorganisms and the dissolved oxygen in the water body. The method of the present disclosure does not use toxic chemical reagents, does not produce waste liquids, has no secondary pollution and less interference factors, and has accurate and stable detection results and high reliability.

In order to achieve the above object, the following technical solution is used in the present disclosure.

A method for detecting ammonia nitrogen content by using a nitrification biological reaction, comprising the following steps:
S1. culture and acclimation of nitrification microorganism membrane, comprising:
continuously conveying an environmental water sample with a temperature of 10-45° C. and a nitrification nutrient solution to a surface of a substrate until a nitrification microorganism membrane is formed on the surface of the substrate,
wherein the nitrification nutrient solution is a mono-component or multi-component solution containing inorganic nitrogen and inorganic carbon; and
S2. ammonia nitrogen detection:
S21. standard curve plotting, comprising:
respectively flowing an ammonia-free water sample and an ammonia-containing standard water sample through the nitrification microorganism membrane obtained in step S1; measuring a dissolved oxygen concentration of an effluent ammonia-free water sample and a dissolved oxygen concentration of an effluent ammonia-containing standard water sample, wherein the dissolved oxygen concentration of the effluent ammonia-free water sample is recorded as DO₁, and the dissolved oxygen concentration of the effluent ammonia-containing standard water sample is recorded as DO₂; calculating a difference value of dissolved oxygen concentration, ADO, between the two effluent water samples; and obtaining a fitting formula according to the ADO and an ammonia nitrogen concentration of the ammonia-containing standard water sample; and
S22. concentration calculation, comprising:
respectively flowing an ammonia-free water sample and an ammonia-containing water sample to be detected through the nitrification microorganism membrane obtained in step S1, detecting and calculating to obtain a difference value of dissolved oxygen concentration, ADO, between the two effluent water samples, and substituting the difference value ADO into the fitting formula obtained in step S21 to calculate an ammonia nitrogen concentration of the ammonia-containing water sample to be detected.

In the present disclosure, the microorganisms in the environmental water sample are continuously colonized and proliferated on the substrate when flowing through the surface of the substrate, and by means of directional stimulation with the nitrification nutrient solution, the metabolism and reproduction of nitrifying bacteria increase, while heterotrophic microorganisms is suppressed.

In the present disclosure, by means of culture with the environmental water sample and acclimation with the nitrification nutrient solution, a nitrification microorganism membrane with strong adaptability to the environment, a stable structure, a high selectivity for ammonia nitrogen, and an ability of efficiently degrading ammonia nitrogen can be obtained, without the need for a highly polluting phosphate buffer solution to maintain the microbial activity.

In the present disclosure, the nitrifying microorganisms have high specificity for ammonia nitrogen, a metabolism and oxygen consumption capacity closely related to the ammonia nitrogen concentration, high accuracy and high sensitivity of ammonia nitrogen detection, and are not easily disturbed by water body chromaticity, suspended matters, and the like. In addition, the activity of the nitrification microorganism membrane reactor can be kept stable for a long term, and the stability of a sensor is good for long-term use.

According to some embodiments of the present disclosure, the environmental water sample and the nitrification nutrient solution are simultaneously and continuously conveyed to the surface of the substrate to form the nitrification microorganism membrane; or

the environmental water sample is first continuously conveyed to the surface of the substrate to obtain a microorganism membrane, and the nitrification nutrient solution is then continuously conveyed to the surface of the obtained microorganism membrane to form a nitrification microorganism membrane.

According to some embodiments of the present disclosure, the environmental water sample comprises an actual water sample containing environmental microorganisms, and the environmental water sample is not defined specially in the present disclosure. For example, the environmental water sample may include at least one of water samples derived from a river, a lake, a sewage treatment plant, domestic sewage, or a fishpond.

According to some embodiments of the present disclosure, the substrate may be at least one of plastic, ceramic, and glass. Furthermore, the substrate may be made from a material which includes but is not limited to at least one of polytetrafluoroethylene, polyurethane, and polyethylene.

According to some embodiments of the present disclosure, the nitrification nutrient solution is a solution containing carbonate ions and ammonium ions.

Preferably, a concentration of carbonate ions in the nitrification nutrient solution is 0.0002-0.06 mg/mL.

Preferably, a concentration of ammonium ions in the nitrification nutrient solution is 0.0002-0.06 mg/mL.

Preferably, the nitrification nutrient solution is a solution containing ammonium carbonate and/or ammonium bicarbonate.

Preferably, the nitrification nutrient solution further comprises nitrite ions.

Preferably, a concentration of nitrite ions in the nitrification nutrient solution is 0.01-10.0 mg/L.

More preferably, the nitrification nutrient solution comprises sodium nitrite.

According to some embodiments of the present disclosure, the ammonia-free water sample, the ammonia-containing standard water sample, the ammonia-containing water sample to be detected, the environmental water sample, and the nitrification nutrient solution are subjected to continuous air saturation.

In the present disclosure, the continuous air saturation refers to aeration of a solution such as an ammonia-free water sample, an ammonia-containing standard water sample, an ammonia-containing water sample to be detected, an environmental water sample, or a nitrification nutrient solution under determined temperature and atmospheric pressure conditions to reach a stable maximum dissolved oxygen content. Generally, the dissolved oxygen in the above air-saturated solution is fixed under given temperature and atmospheric pressure conditions, for example, the dissolved oxygen concentration is stable at about 9.0 mg/L at a temperature of 20° C. and about 7.5 mg/L at a temperature of 30° C. Maintaining a relatively saturated dissolved oxygen concentration can ensure that the nitrification microorganism membrane has enough oxygen concentration during acclimation and culture.

In the solution of the present disclosure, the dissolved oxygen concentration of the ammonia-free water sample, the ammonia-containing standard water sample, the ammonia-containing water sample to be detected, the environmental water sample, or the nitrification acclimation solution is not less than 2 mg/L.

According to some embodiments of the present disclosure, the temperature in step S1 is 10-45° C., including but not limited to 12-45° C., 18-32° C., 25-37° C., 28-35° C., 30° C., 35° C. etc.; preferably 25-37° C.

After culture under these conditions, the cultured microorganisms have a strong adaptability to the environment, a stable structure, a high selectivity for ammonia nitrogen, and an ability of more efficiently degrading ammonia nitrogen.

According to some embodiments of the present disclosure, the ammonia-containing standard water sample in step S2 is an ammonium chloride solution.

According to some embodiments of the present disclosure, an ammonia nitrogen concentration in the ammonium chloride solution is 0-40 mg/L, including but not limited to 0-30 mg/L, 0-25 mg/L, 0-20 mg/L, 0-10 mg/L, 0-8 mg/L, 0-4 mg/L, or 0-2 mg/L.

Within this concentration range, the ammonia nitrogen concentration is linear with the oxygen consumption capacity of the microorganisms.

According to some embodiments of the present disclosure, the flow rates of the environmental water sample and the nitrification nutrient solution in step S1, and the flow rate of each water sample through the nitrification microorganism membrane in step S2 are independently 0.1-10 mL/min, including but not limited to 0.1-8 mL/min, 0.1-5 mL/min, 1-5 mL/min, 1-10 mL/min, 1-8 mL/min, 2-5 mL/min; preferably 2-5 mL/min; more preferably 2-3 mL/min.

The accuracy of the ammonia nitrogen concentration measured at such a flow rate is higher.

According to some embodiments of the present disclosure, the nitrification microorganism membrane obtained in step S1 is filled with tap water and is stored at room temperature before use.

In the present disclosure, the term “ammonia-free water sample” means a water sample free of ammonia nitrogen, and is preferably tap water.

Compared with the prior art, the beneficial effects of the present disclosure are as follows.

In the present disclosure, the nitrifying microorganisms have high specificity for ammonia nitrogen, a metabolism and oxygen consumption capacity closely related to the ammonia nitrogen concentration, high accuracy and high sensitivity of the ammonia nitrogen detection results, and are not easily disturbed by water body chromaticity, suspended matters, and the like. In addition, the activity of the nitrification microorganism membrane reactor can be kept stable for a long term, and the stability of a sensor is good for long-term use. Under the microorganism culture conditions and test conditions of the present disclosure, the relative deviation of the ammonia nitrogen concentration of water as determined by the test meets the requirements and can be minimally 0.55%.

### BRIEF DESCRIPTION OF DRAWINGS

FIG. 1 is a schematic diagram of ammonia nitrogen detection in an embodiment, wherein 1 is a sample to be detected, 2 is a two-position three-way valve, 3 is a flow pipe, 4 is tap water, 5 is a peristaltic pump, 6 is a reactor, and 7 is a dissolved oxygen sensor.

### DETAILED DESCRIPTION

In order to better illustrate the object, technical solution, and advantages of the present disclosure, the present disclosure will be further illustrated below in conjunction with specific examples and accompanying drawings, but the examples do not impose any form of limitation on the present disclosure. Unless otherwise specified, the reagents, methods, and equipment used in the present disclosure are conventional reagents, methods, and equipment in the technical field. Unless otherwise specified, the reagents and materials used in the present disclosure are all commercially available.

The inventive idea of the present disclosure is that: an actual water sample containing environmental microorganisms is used as a water source, and the nitrifying bacteria are directionally screened by means of an acclimation solution containing inorganic carbon and inorganic nitrogen, so as to obtain a microorganism membrane reactor with high nitrifying activity; and a green ammonia nitrogen analysis method is established by using the characteristic of the nitrification microorganism membrane to specifically degrade ammonia and consume oxygen.

### Example 1

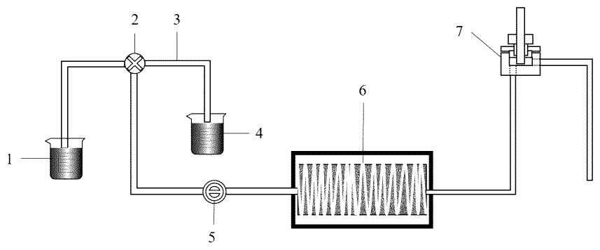

This example provided a method for detecting ammonia nitrogen content by a nitrification biological reaction, and the detection schematic diagram was as shown in FIG. 1. The method comprised the following steps:

S1. Culture and acclimation of a microorganism membrane reactor, comprising:

S11. Preparation of Reactor

A polytetrafluoroethylene tube (with an inner diameter of 2.5 mm and a length of 150 cm) was thoroughly cleaned with water, curled into a spiral shape with a die, and put into an oven at a temperature of 150° C. for heating and shaping to form a polytetrafluoroethylene reactor. The prepared polytetrafluoroethylene reactor was rinsed with a large amount of water, and dried with a nitrogen flow.

S12. Preparation of Environmental Water Sample

An environmental water sample was obtained by filtering a fishpond water sample through a 100-mesh filter.

S13. Preparation of Nitrification Nutrient Solution

9.439 g of (NH₄)₂SO₄, 9.638 g of Na₂CO₃, and 0.382 g of NaNO₂ powder were weighed and put into a 1 L beaker, completely dissolved by adding 800 mL of tap water, then transferred to a 1 L volumetric flask, added tap water to reach the graduation mark, and shaken until uniform to obtain a nitrification nutrient solution stock. The nitrification nutrient solution stock was diluted by tap water for a factor of 100 to obtain a nitrification nutrient solution.

S14. Preparation of Standard Solutions

1) 3.8190 g of ammonium chloride (103° C., dried for 2 h) was accurately weighed and dissolved in tap water, and after being completely dissolved, the solution was made up to 1 L to obtain an ammonia nitrogen stock solution with an ammonia nitrogen concentration of 1000.0 mg/L;

2) 2 mL of the ammonia nitrogen stock solution with a concentration of 1000.0 mg/L was accurately pipetted and placed in a 100 mL volumetric flask, the volume was adjusted to the graduation mark with tap water, and after uniform shaking, an ammonia nitrogen standard intermediate solution with a concentration of 20.0 mg/L was obtained; and

3) 1.25 mL, 2.5 mL, 5.0 mL, 7.5 mL, and 10.0 mL of the ammonia nitrogen intermediate solution with a concentration of 20.0 mg/L were respectively accurately pipetted and placed in 100 mL volumetric flasks, the volume was adjusted to the graduation mark with tap water, and after uniform shaking, ammonia nitrogen standard solutions with concentrations of 0.25 mg/L, 0.5 mg/L, 1.0 mg/L, 1.5 mg/L, and 2.0 mg/L were respectively obtained.

S15. Culture of Microorganism Membrane Reactor

The environmental water sample obtained in S12 was continuously aerated to make it reach an air-saturated state, a peristaltic pump 5 was used as power to continuously introduce the environmental water sample into a polytetrafluoroethylene reactor 6, tap water 4 was introduced into the reactor 6 at a high flow rate every 30 minutes to clean the surface of the microorganism membrane for 30 s, and after the cleaning was completed, the environmental water sample was continuously introduced into the reactor 6 at a low flow rate. The operation was so repeated until the biological oxygen consumption performance of the microorganism membrane reactor 6 reached the expectation. The culture conditions were as follows: the introduction rate of the environmental water sample was 2 mL/min, the introduction rate of the tap water for cleaning was 100 mL/min, and the temperature was 35° C.

S16. Judgment of the Performance of Microorganism Membrane Reactor

During culturing, the change of the dissolved oxygen signal was tracked periodically to feed back the culture state of the microorganism membrane reactor 6. Specifically, tap water 4 continuously flowed through the microorganism membrane reactor 6 as a blank, and the dissolved oxygen concentration DO1 of the effluent was monitored by a dissolved oxygen sensor 7; subsequently, the ammonium chloride standard solution with an ammonia nitrogen concentration of 1.0 mg/L continuously flowed through the microorganism membrane reactor 6, and the dissolved oxygen concentration DO2 of the effluent was monitored by the dissolved oxygen sensor 7; and when ADO (i.e. |DO₁-DO₂|) tended to be stable in several consecutive tests, it could be concluded that the oxygen consumption performance of the microorganism membrane reactor 6 was basically stable. The cultured microorganism membrane reactor was filled with tap water and stored at room temperature before use.

S17. Acclimation of Nitrification Microorganism Membrane Reactor

The microorganism nutrient solution obtained in S13 was continuously aerated to make it reach an air-saturated state, and the peristaltic pump 5 was used as power to continuously introduce the air-saturated nitrification nutrient solution into the microorganism membrane reactor 6 until the performance of the nitrification microorganism membrane reactor 6 reached the expectation. During this period, at intervals, an air-saturated ammonium chloride standard solution with an ammonia nitrogen concentration of 1.0 mg/L or an air-saturated glucose standard solution with a biochemical oxygen demand (BOD) concentration of 8.0 mg/L were tested according to the steps in S16, and the results were as shown in Table 1.

TABLE 1
Monitoring of oxygen consumption intensity of
nitrification microorganism membrane for ammonia
nitrogen and glucose during acclimation

| | Oxygen consumption | Oxygen consumption |
|---|---|---|
| Acclimation | intensity for ammonia | intensity for |
| time (h) | nitrogen (mg/L) | glucose (mg/L) |
| 0 | 0.860 | 0.523 |
| 6 | 1.085 | 0.511 |
| 12 | 1.150 | 0.500 |
| 18 | 1.374 | 0.310 |
| 24 | 1.500 | 0.132 |
| 30 | 1.843 | 0.074 |
| 36 | 2.458 | 0.053 |
| 42 | 2.660 | 0.053 |
| 48 | 2.713 | 0.045 |
| 54 | 2.794 | 0.032 |
| 60 | 2.822 | 0.030 |
| 66 | 2.818 | 0.031 |
| 72 | 2.820 | 0.031 |

It could be seen that the oxygen consumption intensity of the nitrification microorganism membrane reactor for the ammonium chloride standard solution with an ammonia nitrogen concentration of 1.0 mg/L increased from 0.860 mg/L to 2.820 mg/L, whereas the oxygen consumption intensity for the glucose standard solution with a biochemical oxygen demand (BOD) concentration of 8.0 mg/L decreased from 0.523 mg/L to 0.031 mg/L. This indicated that nitrifying bacteria gradually occupy the main part in the nitrification microorganism membrane reactor, and the oxygen consumption of the microorganisms for ammonia was remarkable. As the acclimation continued, the oxygen consumption intensity of the nitrification microorganism membrane reactor for the ammonium chloride standard solution with an ammonia nitrogen concentration of 1.0 mg/L reached a maximum value of 2.822 mg/L at 60 h, whereas the oxygen consumption intensity for the glucose standard solution with a biochemical oxygen demand (BOD) concentration of 8.0 mg/L decreased to 0.030 mg/L, which could be ignored basically.

After 72 hours of continuous acclimation, the above test signal reached stability and the acclimation ended. The acclimatized nitrification microorganism membrane reactor was filled with tap water and stored at room temperature before use.

The acclimation conditions were as follows: the aeration rate of the nitrification nutrient solution was 2 L/min, the test temperature was 35° C., and the flow rate was 2.0 mL/min.

S2. Detection of Ammonia Nitrogen Concentration

S21. Standard Curve Plotting

Tap water 4, as a blank, continuously flowed through the nitrification microorganism membrane reactor 6, and after 10 min, the dissolved oxygen concentration DO1 of the effluent was monitored by a dissolved oxygen sensor 7; and subsequently, the ammonium chloride standard solution with an ammonia nitrogen concentration of 0.25 mg/L continuously flowed through the nitrification microorganism membrane reactor 6, and after 10 min, the dissolved oxygen concentration DO11 of the effluent was monitored by the dissolved oxygen sensor 7, and ΔDO1 (i.e. |DO₁-DO₁₁|) was calculated. According to the above steps, the ammonia nitrogen standard solutions with ammonia nitrogen concentrations of 0.5 mg/L, 1.0 mg/L, 1.5 mg/L, and 2.0 mg/L were tested respectively, and each of the obtained ADO values was recorded. Test conditions: peristaltic pump flow rate 2.0 mL/min, and temperature 35° C.

A curve was plotted with the concentration of the ammonia nitrogen standard solution as the abscissa and the corresponding difference value of dissolved oxygen concentration, ΔDO (microbial oxygen consumption), obtained from the experiment as the ordinate, and after fitting, a linear equation of ammonia nitrogen concentration vs. microbial oxygen consumption ΔDO was obtained: ΔDO=2.824×c[NH₄⁺]−0.052, wherein the linear range of ammonia nitrogen concentration was 0-2.0 mg/L, and the correlation coefficient was 0.9989.

S22. Actual Water Sample Test

Tap water 4 and a sample to be detected 1 were respectively added to two beakers, continuously aerated, and heated to 35° C. in a water bath. According to step S21, firstly, the tap water 4 was flowed through the nitrification microorganism membrane reactor 6 at a flow rate of 2.0 mL/min by using the peristaltic pump 5, and the dissolved oxygen concentration of the effluent was monitored by the dissolved oxygen sensor 7, after 10 minutes, when the dissolved oxygen concentration was stable and no longer changed, the dissolved oxygen concentration DO1 was recorded; the two-position three-way valve 2 was switched, such that the sample to be detected 1 was flowed through the nitrification microorganism membrane reactor 6 at a flow rate of 2.0 mL/min, and the dissolved oxygen concentration dropped rapidly, after 10 minutes, when the dissolved oxygen concentration was stable and no longer changed, the dissolved oxygen concentration DO2 of the effluent was measured by the dissolved oxygen sensor 7. According to the difference value of dissolved oxygen concentrations of the two effluents, the dissolved oxygen consumption ΔDO was calculated, and substituted into the fitting equation of the standard curve, the ammonia nitrogen concentration of the water sample to be detected could be obtained.

The actual water samples were campus sewage, river water, and urban sewage, and the measurement results obtained according to S22 were shown in Table 3.

S3. Verification Experiment

S31. Ammonia nitrogen degradation efficiency of nitrification microorganism membrane reactor

The effluent of the nitrification microorganism membrane reactor was tested for ammonia nitrogen, nitrite nitrogen, nitrate nitrogen, and total nitrogen according to the method in the standard Technical Specifications Requirements for Monitoring of Surface Water and Waste Water (HJ/T 91-2002), and the test results were compared to verify the accuracy (expressed by relative error) of the test method of the present disclosure, wherein ammonia nitrogen degradation rate (%)=(|influent ammonia nitrogen concentration−effluent ammonia nitrogen concentration|)/influent ammonia nitrogen concentration * 100%.

Specifically, the 2 mg/L air-saturated ammonia nitrogen standard solution was continuously aerated, heated to 35° C. in a water bath, and then continuously introduced into the nitrification microorganism membrane reactor 6 at a flow rate of 2 mL/min by using the peristaltic pump 5. The effluent from the reactor 6 was measured, which had an ammonia nitrogen mass concentration of 0.017 mg/L, a nitrite nitrogen mass concentration of 0.014 mg/L, a nitrate nitrogen mass concentration of 1.972 mg/L, and a total nitrogen mass concentration of 2.006 mg/L, and the ammonia nitrogen degradation efficiency of the nitrification microorganism membrane reactor 6 was calculated to be 99.15%.

S32. Information about Microorganisms in Nitrification Microorganism Membrane Reactor

The biological membrane on the inner wall of the nitrification microorganism membrane reactor was taken for high-throughput sequencing to obtain microbial population information, and the results showed that the nitrification microorganism membrane was mainly composed of nitrifying bacteria, wherein the key populations along with the proportions thereof were Nitrosomonadaceae (32.12%), Nitrospiraceae (29.32%), and Nitrososphaeraceae (8.96%), respectively.

S33. Specificity of Nitrification Microorganism Membrane Reactor

For general actual water samples, the mass concentrations of the two water quality indicators, i.e., ammonia nitrogen and biochemical oxygen demand, were maintained at about 1:3 to 1:5. In order to clarify the possible interference of organic matters in the actual water sample, a variety of organic matter solutions with a biochemical oxygen demand concentration of 8.0 mg/L were prepared to explore the specificity of the nitrification microorganism membrane reactor for ammonia.

The preparation method for an organic matter stock solution involved: respectively weighing 2865 mg of glucose, 3412 mg of lactose, 3126 mg of galactose, 3236 mg of sucrose, 2076 mg of glycine, 5675 mg of lysine, 3003 mg of sorbitol, 5030 mg of fumaric acid, 145 mg of benzoic acid, and 4762 mg of citric acid and putting them in 1 L beakers, separately adding 800 mL of tap water to dissolve them completely, then separately transferring them to 1 L volumetric flasks, and the solutions were made up to reach the graduation mark and shaken until uniform.

The preparation method for an organic matter service solution involved: accurately pipetting 4 mL of the organic matter stock solution into a 1000 mL volumetric flask, adjusting the volume to the graduation mark with tap water, and shaking the solution until uniform to obtain an organic matter service solution with a biochemical oxygen demand concentration of 8.0 mg/L.

The preparation method for a mixed organic matter service solution involved: taking 50 mL of each of the glucose, lactose, galactose, sucrose, glycine, lysine, sorbitol, fumaric acid, benzoic acid, and citric acid service solutions and fully mixing them in a 1000 mL beaker.

According to the steps in S22, the dissolved oxygen consumption ΔDO for each organic matter was detected, and the results were detailed in Table 2 below.

TABLE 2
Dissolved oxygen consumption ΔDO for each organic matter

| | Dissolved oxygen difference |
|---|---|
| Organic matter | value ΔDO (mg/L) |
| Glucose | 0.034 |
| Lactose | 0.027 |
| Galactose | 0.030 |
| Sucrose | 0.034 |
| Lysine | 0.094 |
| Glycine | 0.250 |
| Sorbitol | 0.031 |
| Fumaric acid | 0.038 |
| Benzoic acid | 0.050 |
| Citric acid | 0.029 |
| Mixed organic matters | 0.059 |

Compared with the response of the nitrification microorganism membrane reactor to the 1 mg/L ammonia nitrogen standard solution, ΔDO=2.722 mg/L, it could be seen that the nitrification microorganism membrane reactor obtained by this method had very little interference response to organic matters.

### Comparative Example 1

This comparative example provided a method for detecting ammonia nitrogen content by using a nitrification biological reaction. The detection was carried out with reference to the steps of Example 1, and the difference from Example 1 lay in that:

Step S1 was carried out only to step S16, and the obtained microorganism membrane reactor was not subjected to acclimation with the nitrification nutrient solution (i.e., step S17 was not carried out). The ammonia nitrogen measurement results were shown in Table 3.

Comparing Example 1 with Comparative Example 1, it could be seen that the accuracy of the measurement of the actual water sample with the microorganism membrane reactor acclimated with the nitrification nutrient solution was significantly higher than that with the microorganism membrane reactor not acclimated with the nitrification nutrient solution. The reason was that the microorganism membrane reactor not acclimated with the nitrification nutrient solution might contain some heterotrophic microorganisms, which could take organic matters in the actual water sample for oxygen consumption metabolism; therefore, the measured oxygen consumption value was higher than the actual value, and as a result, the obtained ammonia nitrogen concentration would be significantly higher than that measured by the national standard method.

### Example 2

This example provided a method for detecting ammonia nitrogen content by using a nitrification biological reaction. The detection was carried out with reference to the steps of Example 1, and the difference from Example 1 lay in that:
in step S11, the selected microorganism membrane culture substrate was a polyurethane hose with an inner diameter of 2.0 mm and a length of 100 cm;
in step S12, the environmental water sample was obtained by filtering sewage from a sewage treatment plant through a 100-mesh screen; and
in step S17, the acclimation conditions were as follows:
the aeration rate of the nitrification nutrient solution was 5 L/min, the test temperature was 30° C., and the test flow rate was 3.0 mL/min; and after 54 h of continuous acclimation of the microorganism membrane reactor, the oxygen consumption intensity of the nitrification microorganism membrane reactor for the ammonium chloride standard solution with an ammonia nitrogen concentration of 1.0 mg/L reached a stable value, i.e., approximately 2.628±0.122 mg/L.

The linear equation of the ammonia nitrogen standard solution obtained in this example was ΔDO=2.633 xc[NH₄⁺]−0.017, wherein the linear range of ammonia nitrogen concentration was 0-2.0 mg/L, and the correlation coefficient was 0.9988.

The ammonia nitrogen detection results were shown in Table 3.

### Example 3

This example provided a method for detecting ammonia nitrogen content by using a nitrification biological reaction. The detection was carried out with reference to the steps of Example 1, and the difference from Example 1 lay in that:
in step S11, the selected microorganism membrane culture substrate was a polyethylene hose with an inner diameter of 2.5 mm and a length of 200 cm;
in step S12, the environmental water sample was obtained by filtering river water through a 100-mesh screen;
in step S13, the preparation of the nitrification nutrient solution was as follows:
3.819 g of NH₄Cl, 7.638 g of NaHCO₃, and 0.382 g of NaNO₂ powder were weighed and put into a 1 L beaker, 800 mL of tap water was added for complete dissolution, the solution was then transferred to a 1 L volumetric flask, tap water was added to reach the graduation mark, and shaken until uniform to obtain a nitrification nutrient solution stock; and the nitrification nutrient solution stock was diluted by tap water for a factor of 100 to obtain a nitrification nutrient solution; and
in step S17, the acclimation conditions were as follows:
the aeration rate of the nitrification nutrient solution was 3 L/min, the test temperature was 25° C., and the test flow rate was 3.0 mL/min; and after 78 h of continuous culture of the microorganism membrane reactor, the oxygen consumption intensity of the nitrification microorganism membrane reactor for the ammonium chloride standard solution with an ammonia nitrogen concentration of 1.0 mg/L reached a stable value, i.e., approximately 1.256±0.137 mg/L.

The linear equation of the ammonia nitrogen standard solution obtained in this example was ΔDO=1.277×c[NH₄⁺]−0.032, wherein the linear range of ammonia nitrogen concentration was 0-2.0 mg/L, and the correlation coefficient was 0.9972.

The ammonia nitrogen detection results were shown in Table 3.

### Example 4

This example provided a method for detecting ammonia nitrogen content by using a nitrification biological reaction. The detection was carried out with reference to the steps of Example 1, and the difference from Example 1 lay in that:
in step S11, the selected microorganism membrane culture substrate was a polyurethane hose with an inner diameter of 2.0 mm and a length of 150 cm;
in step S12, the environmental water sample was obtained by filtering a school domestic sewage water sample through a 100-mesh screen;
in step S13, the preparation of the nitrification nutrient solution was as follows:
3.819 g of NH₄Cl and 7.638 g of NaHCO₃ powder were weighed and put into a 1 L beaker, 800 mL of tap water was added for complete dissolution, the solution was then transferred to a 1 L volumetric flask, tap water was added to reach the graduation mark, and shaken until uniform to obtain a nitrification nutrient solution stock; and the nitrification nutrient solution stock was diluted by tap water for a factor of 100 to obtain a nitrification nutrient solution; and
in both the culture conditions of step S15 and the acclimation conditions of step S17, the temperature was 30° C.

The linear equation of the ammonia nitrogen standard solution obtained in this example was ΔDO=1.911×c[NH₄⁺]−0.037, wherein the linear range of ammonia nitrogen concentration was 0-2.0 mg/L, and the correlation coefficient was 0.9999.

Tap water and a nitrogen-containing water sample to be detected were respectively flowed through the nitrification microorganism reaction membrane, measured and calculated to obtain the difference value of dissolved oxygen concentration, ΔDO, between the two effluents water samples, and the difference value was substituted into the obtained fitting formula to calculate the ammonia nitrogen concentration in the ammonia-containing water sample to be detected. The test results were shown in Table 3.

The 2 mg/L air-saturated ammonia nitrogen standard solution was continuously aerated, heated to 30° C. in a water bath, and then continuously introduced into the nitrification microorganism membrane reactor 6 at a flow rate of 2 mL/min by using the peristaltic pump 5. The mass concentrations of ammonia nitrogen, nitrite nitrogen, nitrate nitrogen, and total nitrogen of the effluent from the nitrification microorganism membrane reactor were measured to be 0.123 mg/L, 0.011 mg/L, 1.803 mg/L, and 2.003 mg/L, respectively, and the ammonia nitrogen degradation efficiency of the microorganism membrane reactor was calculated to be 93.85%.

The nitrification microorganism membrane reactor obtained in this example was mainly composed of nitrifying bacteria.

### Example 5

This example provided a method for detecting ammonia nitrogen content by using a nitrification biological reaction. The detection was carried out with reference to the steps of Example 2, and the difference from Example 2 lay in that:
in step S13, the preparation of the nitrification nutrient solution was as follows:
3.819 g of NH₄Cl and 7.638 g of NaHCO₃ were weighed and put into a 1 L beaker, 800 mL of tap water was added for complete dissolution, the solution was then transferred to a 1 L volumetric flask, tap water was added reach the graduation mark, and shaken until uniform to obtain a nitrification nutrient solution stock; and the nitrification nutrient solution stock was diluted by tap water for a factor of 50 to obtain a nitrification nutrient solution.
Steps S15, S16, and S17 were combined, and the culture and acclimation of the microorganism reaction membrane were carried out simultaneously. The specific steps were as follows:
The nitrification nutrient solution obtained in S13 and the environmental water sample obtained in S12 were mixed at 1:1 to obtain a culture acclimation solution. The culture acclimation solution was continuously aerated to make it reach an air-saturated state, and the peristaltic pump 5 was used as power to continuously introduce the culture acclimation solution into the reactor 6 until the performance of the nitrification microorganism membrane reactor 6 reached the expectation. The culture test conditions were as follows: the aeration rate of the culture solution was 3 L/min, the test temperature was 30° C., and the test flow rate was 3.0 mL/min. After 60 h of continuous culture of the nitrification microorganism membrane reactor, the oxygen consumption intensity of the nitrification microorganism membrane reactor for the ammonium chloride standard solution with an ammonia nitrogen concentration of 1.0 mg/L reached a stable value, i.e., approximately 1.112±0.137 mg/L.

The linear equation of the ammonia nitrogen standard solution obtained in this example was ΔDO=1.1316×c[NH₄⁺]+0.0067, wherein the linear range of ammonia nitrogen concentration was 0-1.75 mg/L, and the correlation coefficient was 0.9899.

Tap water and a nitrogen-containing water sample to be detected were respectively flowed through the nitrification microorganism reaction membrane, measured and calculated to obtain the difference value of dissolved oxygen concentration, ΔDO, between the two effluent water samples, and the difference value was substituted into the obtained fitting formula to calculate the ammonia nitrogen concentration of the ammonia-containing water sample to be detected. The test results were shown in Table 3.

### Comparative Example 2

This comparative example provided a method for detecting ammonia nitrogen content by using a nitrification biological reaction. The detection was carried out with reference to the steps of Example 5, and the difference from Example 5 lay in that the test temperature was 20° C.

### Comparative Example 3

This comparative example provided a method for detecting ammonia nitrogen content by using a nitrification biological reaction. The detection was carried out with reference to the steps of Example 5, and the difference from Example 5 lay in that the test temperature was 40° C.

### Comparative Example 4

This comparative example provided a method for detecting ammonia nitrogen content by using a nitrification biological reaction. The detection was carried out with reference to the steps of Example 5, and the difference from Example 5 lay in that: the culture acclimation solution was prepared from the following raw materials: 0.5 g of ammonium sulfate, 0.3 g of sodium chloride, 0.03 g of ferrous sulfate, 1 g of sodium dihydrogen phosphate, 0.03 g of magnesium sulfate, and 7.5 g of calcium chloride, which were added to water to prepare a 1000 mL culture acclimation solution.

### Example 6

This example provided a method for detecting ammonia nitrogen content by using a nitrification biological reaction. The detection was carried out with reference to the steps of Example 5, and the difference from Example 5 lay in that:

in step S11, the selected microorganism membrane culture substrate was a polyethylene hose with an inner diameter of 2.5 mm and a length of 200 cm;

in step S13, the preparation of the nitrification nutrient solution was as follows:

3.819 g of NH₄Cl, 7.638 g of NaHCO₃, and 0.382 g of NaNO₂ powder were weighed and put into a 1 L beaker, 800 ml of tap water was added for complete dissolution, the solution was then transferred to a 1 L volumetric flask, tap water was added to reach the graduation mark, and shaken until uniform; and the nitrification nutrient solution stock was diluted by tap water for a factor of 50 to obtain a nitrification nutrient solution.

Similarly, steps S15, S16, and S17 were combined, and the culture and acclimation of the microorganism reaction membrane were carried out simultaneously. The specific steps were different from those in Example 5 in that in the culture test conditions, the test temperature was 35° C.

The linear equation of the ammonia nitrogen standard solution obtained in this example was ΔDO=2.783×c[NH₄⁺]−0.015, wherein the linear range of ammonia nitrogen concentration was 0-2.0 mg/L, and the correlation coefficient was 0.9996.

Tap water and a nitrogen-containing water sample to be detected were respectively flowed through the nitrification microorganism reaction membrane, measured and calculated to obtain the difference value of dissolved oxygen concentration, ΔDO, between the two effluent water samples, and the difference value was substituted into the obtained fitting formula to calculate the ammonia nitrogen concentration of the ammonia-containing water sample to be detected. The test results were shown in Table 3.

The 2 mg/L air-saturated ammonia nitrogen standard solution was continuously aerated, heated to 35° C. in a water bath, and then continuously introduced into the nitrification microorganism membrane reactor 6 at a flow rate of 3 mL/min by using the peristaltic pump 5. The mass concentrations of ammonia nitrogen, nitrite nitrogen, nitrate nitrogen, and total nitrogen in the effluent from the reactor was measured to be 0.019 mg/L, 0.018 mg/L, 1.969 mg/L, and 2.008 mg/L, respectively, and the ammonia nitrogen degradation efficiency of the nitrification microorganism membrane reactor was calculated to be 99.05%.

The nitrification microorganism membrane reactor obtained in this example was mainly composed of nitrifying bacteria.

Test Results

The test results of the test methods of the above examples and comparative examples were detailed in Table 3. In the present disclosure, the nitrogen-containing water sample to be detected used in each example or comparative example was also tested according to the method described in the standard ISO 7150-1-1984, and the test results were compared to verify the accuracy (expressed by relative error) of the testing method of the present disclosure, wherein the relative error (%)=(|the result of the present disclosure−the standard result|)/the standard result * 100%.

TABLE 3

| | ISO 7150-1-1984 | Detection result of | Relative |
|---|---|---|---|
| | detection results | the present disclosure | error |
| Sample | (mg/L) | (mg/L) | (%) |
| Example 1-1 | 1.567 | 1.614 | 2.999 |
| Example 1-2 | 0.277 | 0.285 | 2.888 |
| Example 1-3 | 1.101 | 1.107 | 0.545 |
| Example 2 | 1.717 | 1.805 | 5.125 |
| Example 3 | 0.912 | 0.893 | 2.083 |
| Example 4-1 | 1.884 | 1.993 | 5.786 |
| Example 4-2 | 1.629 | 1.701 | 4.420 |
| Example 4-3 | 0.639 | 0.624 | 2.347 |
| Example 5 | 0.45 | 0.43 | 4.44 |
| Example 6-1 | 0.769 | 0.754 | 1.951 |
| Example 6-2 | 0.917 | 0.905 | 1.309 |
| Example 6-3 | 1.347 | 1.394 | 3.489 |
| Comparative | 0.641 | 0.842 | 31.357 |
| Example 1-1 | | | |
| Comparative | 0.912 | 1.324 | 45.175 |
| Example 1-2 | | | |
| Comparative | 0.830 | 0.945 | 13.855 |
| Example 1-3 | | | |
| Comparative | 0.13 | 0.11 | 15.38 |
| Example 2 | | | |
| Comparative | 0.65 | 0.75 | 15.38 |
| Example 3 | | | |
| Comparative | 1.19 | 1.28 | 7.56 |
| Example 4 | | | |

Note:
“Example 1-1”, “Example 1-2” and “Example 1-3” indicate that Example 1 was conducted three times in parallel. Similar expressions, such as “Example 4-1”, “Example 4-2” and “Example 4-3” have similar mean.

As could be seen from the above results, the test results obtained by the test method of the present disclosure had high accuracy; moreover, the composition of the culture solution and the culture temperature had certain influence on the accuracy of the test results.

Finally, it should be noted that the above embodiments are only used to illustrate the technical solution of the present disclosure and not to limit the scope of protection of the present disclosure. Although the present disclosure has been illustrated in detail with reference to preferred embodiments, it should be understood by those of ordinary skill in the art that the technical solution of the present disclosure can be modified or replaced by equivalents without departing from the essence and scope of the technical solution of the present disclosure.

1. A method for detecting ammonia nitrogen content by a nitrification biological reaction, comprising the following steps:
S1. culture and acclimation of nitrification microorganism membrane, comprising:
continuously conveying an environmental water sample with a temperature of 10-45° C. and a nitrification nutrient solution to a surface of a substrate until a nitrification microorganism membrane is formed on the surface of the substrate,
wherein the nitrification nutrient solution is a mono-component or multi-component solution containing inorganic nitrogen and inorganic carbon; and
S2. ammonia nitrogen detection:
S21. standard curve plotting, comprising:
respectively flowing an ammonia-free water sample and an ammonia-containing standard water sample through the nitrification microorganism membrane obtained in step S1;
measuring a dissolved oxygen concentration of an effluent ammonia-free water sample and a dissolved oxygen concentration of an ammonia-containing standard water sample, wherein the dissolved oxygen concentration of the effluent ammonia-free water sample is recorded as DO₁, and the dissolved oxygen concentration of the effluent ammonia-containing standard water sample is recorded as DO₂; calculating a difference value of dissolved oxygen concentration, ΔDO, between the two effluent water samples; and obtaining a fitting formula according to the ΔDO and an ammonia nitrogen concentration of the ammonia-containing standard water sample; and
S22. concentration calculation, comprising:
respectively flowing an ammonia-free water sample and an ammonia-containing water sample to be detected through the nitrification microorganism membrane obtained in step S1, detecting and calculating to obtain a difference value of dissolved oxygen concentration, ΔDO, between the two effluent water samples, and substituting the difference value into the fitting formula obtained in step S21 to calculate an ammonia nitrogen concentration of the ammonia-containing water sample to be detected.

2. The method for detecting ammonia nitrogen content by using the nitrification biological reaction according to claim 1, wherein the environmental water sample and the nitrification nutrient solution are simultaneously and continuously conveyed to the surface of the substrate to form the nitrification microorganism membrane; or
the environmental water sample is first continuously conveyed to the surface of the substrate to obtain a microorganism membrane, and the nitrification nutrient solution is then continuously conveyed to the surface of the obtained microorganism membrane to form the nitrification microorganism membrane.

3. The method for detecting ammonia nitrogen content by using the nitrification biological reaction according to claim 1, wherein the environmental water sample comprises at least one of water samples derived from a river, a lake, a sewage treatment plant, domestic sewage, or a fishpond.

4. The method for detecting ammonia nitrogen content by using the nitrification biological reaction according to claim 1, wherein the nitrification nutrient solution is a solution containing carbonate ions and ammonium ions.

5. The method for detecting ammonia nitrogen content by using the nitrification biological reaction according to claim 4, wherein the nitrification nutrient solution further comprises nitrite ions.

6. The method for detecting ammonia nitrogen content by using the nitrification biological reaction according to claim 1, wherein the ammonia-free water sample, the ammonia-containing standard water sample, the ammonia-containing water sample to be detected, the environmental water sample, and the nitrification nutrient solution are subjected to continuous air saturation.

7. The method for detecting ammonia nitrogen content by using the nitrification biological reaction according to claim 1, wherein the temperature in step S1 is 25-37° C.

8. The method for detecting ammonia nitrogen content by using the nitrification biological reaction according to claim 1, wherein the ammonia-containing standard water sample in step S2 is an ammonium chloride solution.

9. The method for detecting ammonia nitrogen content by using the nitrification biological reaction according to claim 8, wherein an ammonia nitrogen concentration of the ammonium chloride solution is 0-40 mg/L.

10. The method for detecting ammonia nitrogen content by using the nitrification biological reaction according to claim 1, wherein flow rates of the environmental water sample and the nitrification nutrient solution in step S1 are independently 0.1-10 mL/min.

11. The method for detecting ammonia nitrogen content by using the nitrification biological reaction according to claim 1, wherein a flow rate of each water sample flowing through the nitrification microorganism membrane in step S2 is independently 0.1-10 mL/min.

12. The method for detecting ammonia nitrogen content by using the nitrification biological reaction according to claim 10, wherein flow rates of the environmental water sample and the nitrification nutrient solution in step S1 are independently 2-3 mL/min.

13. The method for detecting ammonia nitrogen content by using the nitrification biological reaction according to claim 11, wherein the flow rate of each water sample flowing through the nitrification microorganism membrane in step S2 is independently 2-3 mL/min.
